(19) United States
(12) Patent Application Publication
(10) Pub. No.: US 2023/0162849 A1
(43) Pub. Date: May 25, 2023

(54) Method for Simulating and Sensing the Likelihood of Failure During Operation of a Medical Product, and Data System for Storing and Transmitting Same

(71) Applicant: W.O.M. World of Medicine GmbH, Berlin (DE)

(72) Inventors: Erika Mase, Berlin (DE); Julia Grundmann, Berlin (DE); Christian Hunger, Oranienburg (DE); Jan-Hendrik Carstens, Berlin (DE); Henrik Stier, Berlin (DE)

(73) Assignee: W.O.M. World of Medicine, Berlin (DE)

(21) Appl. No.: 17/917,711

(22) PCT Filed: Apr. 9, 2021
(86) PCT No.: PCT/DE2021/000068

§ 371 Date: Oct. 7, 2022

(30) Foreign Application Priority Data
Apr. 9, 2020 (DE) 10 2020 002 233.2

(51) Int. Cl. G16H 40/40

(52) U.S. Cl. CPC G16H 40/40

(57) ABSTRACT

Subject matter of the invention is a medical-technical system comprising a sensor system for directly or indirectly sensing all conditions relevant to a failure of the subassemblies of the medical-technical system, and a simulation module, which simulates, based on the sensor data and selected applications of the medical-technical system, the actual operating parameter values, the history of use, and the effects caused by the indication with the aid of the known wear properties of the components, the application-dependent stability of the components in the medical-technical system, and outputs it in a complete or simplified form or as instructions for repair.

### SUBJECT MATTER OF THE INVENTION

The invention relates to a medical-technical system comprising a sensor system for directly or indirectly sensing all conditions relevant to a failure of the subassemblies of the medical-technical system, and a simulation module, which simulates, based on the sensor data and selected applications of the medical-technical system, the actual operating parameter values, the history of use, and the effects caused by the indication, with the aid of known wear properties of the components, the application-dependent stability of the components in the medical-technical system, and outputs it in a complete or simplified form or as instructions for repair. Preferably, the sensed data are collected in a data system and transmitted to a replacement device used at the same location and having the same behavior of usage.

### PRIOR ART

In medical technology, devices are not usually monitored by sensors with regard to failure risks. Preventive maintenance of devices is usually based on the precautionary replacement of critical components threatened with failure during periodical maintenance or safety checks.

When unexpected increases of failures of components occur, then, according to the prevailing legal regulations in the EU and the USA, the manufacturer shall inform the clients, in some circumstances interdict the use, and replace the conspicuous modules of the devices on the client's site.

There are some systems in medical technology that offer such sensing by sensors, however to a not very large extent.

The document U.S. Pat. No. 9,468,447 describes an orthopedic system, which is provided with data collection modules for recording performance features and data analysis modules for evaluating the measurement values. Based on the measurement values, the data analysis modules analyze, with the aid of suitable algorithms, the wear situation and output maintenance or repair recommendations. The consideration of indication-dependent wear is not described, and a reliable prognosis for the future/likelihood of failure depending on the indication is not disclosed.

Outside medical technology, preventive maintenance or sensor-based diagnosis can be found more frequently, however, here too, not to such a large extent as disclosed in this invention.

The document DE 102009049931 describes a diagnosis and maintenance device for switchgear with a data processing device and at least one internal interface device, which is in communication with a connected device ready for communication in the respective switchgear and queries and/or processes the diagnosis and maintenance information and/or status information thereof and provides and/or outputs and/or displays them as usable and/or machine-readable information to be retrieved. It is not described to evaluate an application-dependent development of the wear, and there is no reliable statement about the application-dependent stability of the components.

The document WO 2006/034852 describes methods for the diagnosis of technical devices disposed within an industrial installation of the processing industry, especially pumps and valves, wherein disturbance variables that affect the devices and influence the service life thereof are detected by means of sensory technology, the data thereof being processed in a simulative manner in order to determine the expected service life of each device, wherein various maintenance instructions are stored in an expert unit, and are output along with the expected remaining trouble-free operating time of the industrial installation when a lower service life threshold value of one or several devices has been reached. It is proposed a diagnosis within an industrial installation, wherein, based on disturbance variables that affect the devices and influence the service life thereof, are detected by means of sensory technology, and the service life to be expected is determined by means of simulation, but the consideration of indication-dependent wear is not described.

The document EP 1836576 describes a method for deciding the time for the replacement of a vacuum pump based on its current performance assessment results. For this purpose, a comparison of the currently evaluated diagnostics analysis results and the initial (or reference) data set is shown, with the aim to enable statements about the necessity of maintenance of the pump, based on pump performance indicators. It is proposed to monitor the performance parameters for determining the necessity of maintenance, a future and application-dependent prognosis of the wear at a later time is, however, not described.

The invention further comprises a data system for transmitting the information already detected in the device to a replacement device provided at the same location and in the same context. For this, there are several solutions in the context of the transmission of information.

The document EP 0497041 describes an infusion pump set for configuring a single infusion pump, in order to mimic the operation of a pump from a plurality of dedicated infusion pumps, the set comprising:
a housing means to be positioned adjacent to a patient for delivering a liquid to the patient;
a reservoir means coupled to the housing for holding the liquid;
a delivery means for delivering the liquid to the patient from the reservoir;
an input means for inputting patient-specific information;
a microprocessor means for controlling the delivery means; and
a memory module that can be selected from a set of memory modules and that contains a program describing a specific delivery profile characteristic of a respective dedicated pump,
   the specially selected memory module being coupled to the microprocessor, and the respective control program for the delivery means being read, and the delivery means being operated to emulate the respectively selected pump.

There is described a control module of a pump, in which several sets of discrete control parameters can be recorded. A manipulation/adaptation of the pump characteristic by means of variation of the control commands is not described, a simulation of the response behavior is not performed, and a matching pump characteristic of different pumps is not generated.

The document EP 2015804 describes an infusion set for administering a medical liquid comprising a data memory for exchanging data with an administration device, wherein the information present on the data memory serves for determining deviations from the treatment-conforming operating status and, in the case of a non-treatment-conforming operating status, a treatment-conforming operating status can be effected in an automated manner or via assistance by the pump wearer. A manipulation/adaptation of the pump characteristic by means of variation of the control commands is not described, a simulation of the response behavior is not performed, and a matching pump characteristic of different pumps is not generated.

Further prior art is described in the documents WO 2006/110246 and EP 3557589 A1.

### SOLUTION ACCORDING TO THE INVENTION

It is therefore the object to sense and display the likelihood of failure of medical-technical devices and the components thereof. For achieving this object, the parameters of use of the typical device applications are sensed and used as a basis for further calculations.

For achieving this object, the invention teaches
a method for sensing and displaying the likelihood of failure of a medical-technical device,
wherein the medical-technical device comprises at least one wear-prone component,
at least one sensor for sensing the operating parameters of the wear-prone component,
at least one memory for storing the operating parameters, the memory comprising at least one operating parameter limit,
at least one computer for calculating the likelihood of failure and at least one display unit,
characterized by that the computer compares the operating parameters of the medical-technical device to stored operating parameter limit data and displays the result in the form of a likelihood of failure and/or a remaining usable life and/or a number of remaining usable cycles,
and medical-technical devices for carrying out such methods.

First of all, the solution according to the invention provides performing, by using known component behavior and sensor data, a simulation of the complete system, by which it is determined whether the components survive the intended use without failure with a predetermined certainty. Further, according to the invention, the simulation is performed beforehand outside the device, and the results of the simulation are assigned, based on determined parameters, to the actual scenario of use, and the result of the assigned simulation is used for determining the risk of failure of the components.

The medical-technical device may be, for instance, a liquid pump for laparoscopy, arthroscopy, hysteroscopy, uroscopy, or an insufflation device.

Such medical-technical devices typically contain components that are subjected to considerable wear, such as, for instance, the motors of peristaltic pumps or the gas pumps of the insufflators. Another example is the vacuum pump of an insufflation device. For calculation of the likelihood of failure, therefore, first, the details of use have to be detected, in order to sense or simulate the wear.

In the simplest case, the sensor may be a clock that counts down (operating hours meter), or also the detection of certain actions with a time stamp and a calculation of the duration of use, which may occur, e.g., directly after sensing.

The sensor may however also represent the detection of the operating current of an electric motor, wherein the operating current relative to the rotating speed permits a statement about the already existing wear of the motor.

Further sensors may be: measuring means for flow measurement, which allow, in combination with the parameters of the control of pumps, a statement about the wear of these pumps, by comparing default and achieved effect.

For instance, the flow of fluids as a function of the consumed electrical power of the pump can be used as a measure for the wear properties. Alternatively, a pressure sensor for pressure measurement of the maximum achievable pressure as a function of the rotating speed of the pump can be used. Another possibility for the determination of the wear properties is the measurement of imbalances, which can be obtained by measurement of the vibrational movement of the motor (e.g., vertically to the axis of rotation). The wear can, however, also be determined acoustically by measurement of the noise during operation. With a microphone, the sound level during operation can be measured. By the spectral analysis of the sound signal, more precise identification of the wearing part can also be enabled (e.g., for pump systems with multiple axes of rotation, as in peristaltic pumps).

More complex in the evaluation is, for instance, the measurement of the dynamics of the fluid flow or of the pressure rise. Another alternative is the measurement of the operating temperature of the pump. The sensors to be used for the respectively selected measurement process are well known to those skilled in the art and are commercially available in many variants so that here no further explanation is required.

For carrying out the method, the medical-technical device includes a memory, in which the data measured by means of the sensor (or the sensors) are stored. When the device comprises a communication interface, the data can be transmitted periodically or upon request via the same to other memory devices. These may be, e.g., data processing and storing devices of a hospital, a doctor's office, or the cloud of the device manufacturer.

For the calculation of the likelihood of failure, first, the load limit of the respective component needs to be known and stored in a memory. This may be an internal memory of the device, but also an external memory, such as, e.g., the manufacturer's cloud.

In the simplest case, a manufacturer can determine, for instance, by corresponding tests that, for instance, a certain component can be used with a likelihood of 95% for more than 1,000 operating hours, before it fails. When the sensor is an operating hours meter, a simple display of the remaining “safe” number of hours may be used. Continuing with this example: When the component was already used for 750 hours, there is a likelihood of 95% that the component can be used for another 250 hours and should be maintained or replaced when 1,000 hours are reached.

Considering the behavior of usage in the calculation of the deadline until component failure can be carried out, for instance, as follows: When a usage is weekly on 1 day for 6 h, then the calculation algorithm simulates in 6-h steps and outputs the number of the week, until, e.g., a confidence value of 95% of the expected lifetime (lifetime limit or operating parameter limit that is known and stored in the data system) of a critical component is reached as a threshold.

In the medical-technical devices according to the invention, the wear is typically not only dependent on the duration of use, but also on the intensity of use. Especially in pump systems, the wear often depends on the pressure that is generated by means of the pump. For instance, a liquid pump can be used with a likelihood of 95% for more than 1,000 operating hours, when a pressure of 35 mm Hg is generated, but only for 500 operating hours when a pressure of 70 mm Hg is generated.

A system according to the invention detects, therefore, preferably not only the duration of use, but also the intensity of use and calculates therefrom the likelihood of failure.

It is understood that the real usage of the medical-technical devices according to the invention depends on a plurality of factors. There are hospitals or doctor's offices that are specialized for certain applications, where the devices are stressed more or less uniformly concerning the duration of use and intensity of use. There are, however, also hospitals or doctor's offices, where the different applications of the devices performed there are very different. For this reason, it is preferred, according to the invention, to record individually every single usage for each device and to use this in the calculation.

In this way, the actual indication is taken into account in the simulation, and known or formerly recorded durations of use are also taken into account in this indication. Feedback of the thus determined remaining usable life (e.g., the calculated number of weeks) may occur, beginning from a threshold value to be defined, via the user interface, e.g., as a display with the specification of the individual (calculated from previous usage) remaining usable life or LED signal. Further, beginning from another threshold value to be defined, the initiation of a preventive service for the preventive exchange of components may be made via an optical or acoustic message or a communication interface in another system (purchasing system in the hospital or at the manufacturer). The transmitted data may include service information with the specification of the device ID, the respective component, and the concrete simulated remaining lifetime, the duration of operation, and the remaining usable life indicated to the user.

According to the invention, this information can be indicated regularly to the user or via the communication interface, at a threshold value, and upon request. The form of the information from the device may be made as an indication on the display, via email, SMS, as acoustic output (voice or sound signal), and by communication according to a protocol to other systems (purchase planning system, hospital information system (HIS), an OP planning system, etc.).

Further is claimed a data system comprising at least:
for every failure-relevant condition, an operating parameter limit value, where the functionality cannot be guaranteed anymore,
the possible operating conditions or applications (indications),
the previous effects on the respective subassemblies depending on the indications.

Such a data system according to the invention allows the transmission of further device parameters (e.g., a usage profile of the device) to another medical-technical system with the same or similar design. These further device parameters may be safety-relevant parameters, such as, e.g., the generated maximum pressure or the maximum flow rate. A user whose medical-technical device provides, for instance, upon request a certain gas flow (e.g., 20 l/min), could be surprised, when the replacement device supplies a higher gas flow. Part of the data set transmitted to the replacement device may be, therefore, the maximum flow rate of the previous device. The user can then continue working with the replacement device in the new device as usual. The transmitted usage profile, if applicable, of the device may also include further device-specific data, for instance, a certain control characteristic, also how fast or slow the device responds, for instance, to disturbances (e.g., pressure drop by intra-operative leakage). The usage of other parameters for the device behavior is also covered by the invention.

A feasible point of time for the calculation and display of the likelihood of failure or remaining usable life and/or remaining usable cycles is the activation of the device (pre-OP, i.e., before an application) and at the end of a procedure (post-OP, after an application), which the device can recognize by certain events, such as the removal of accessories.

It is obvious, for those skilled in the art, that in case of maintenance or replacement of a wear-prone component, the stored data can correspondingly be adapted, for instance by resetting the detected operating hours to zero with replacement by a new component of identical design and/or change of the duration of use when using a higher-value component.

The collected and evaluated data according to the invention can be transmitted to other devices, for instance to replacement devices of identical design.

Transmission of the collected data to another device can generate, according to the invention, if the receiving system does not have an identical design, an identical system response to the user interaction, that is, emulate the device behavior, influence the response behavior of systems (such as response time). The transmission occurs such that existing pre-information about the technical behavior is transmitted from a transmitting/old to a replaced/receiving device. This may comprise the enabled indications (if, e.g., certain usage modes are blocked or are only usable after payment), the recorded user behavior, and performed adaptations based on the user behavior and calculated/simulated values.

The access to these data may take place in the device via a menu item to be selected, and/or automatically at fixed information times, this possibly only comprising a selection, and full data access being offered as a selection item, and/or via a communication interface or network connection (Ethernet, WLAN), and/or by means of a special interface such as, e.g., inputting into a memory device when the latter is connected, especially via USB when a corresponding device is plugged in. The data may optionally be encrypted, and access occurs via an entitlement authenticated by a password or the like.

The method according to the invention enables preventive maintenance of medical-technical devices. The method provides the user (e.g., doctor), the operator (e.g., the hospital), and/or the device manufacturer with data allowing them to maintain or replace critical components before a failure occurs. An intra-operative failure of such a component is to be prevented for understandable reasons. By the method according to the invention, it is secured that the medical-technical devices are not (or cannot be) further used, when it can be expected that further usage exceeds the limit values. By the method according to the invention, it is furthermore possible to enable, in due time before reaching the parameter limits, maintenance or replacement.

### EXAMPLE

A possible sequence for a distention pump for expanding a body cavity could be as follows:
Loading the tube set and/or removing the tube set

(Alternatively: with user input for turning the device on and off).
Simulation of the likelihood of failure of the components using the behavior of usage/operating parameter information (also historically), which is stored in a data structure (possibly also in a cloud). Further, actually selected usage scenarios (indications for multi-pumps) are taken into account for the simulation. The basis for the simulation is a component behavior (stability) previously determined under certain usage scenarios. This information may be provided by the manufacturer of the components—such as the likelihood of failure or duration of use (MTBF). The simulation takes place before the usage of the device (outside or inside the device) and determines the remaining lifetime or information about the required replacement before the intended usage, based on the applied behavior.
The simulation uses sensor data, such as, e.g., the motor current necessary for a certain rotating speed in a known usage phase (e.g., after insertion of the tube the turns for filling the tube), in order to determine therefrom the wear of the drive unit.
Transfer of simulation results to a display device. When certain threshold values are reached, the simulation results are also transferred to further systems, e.g., HIS/purchasing or technical service of the manufacturer.
When the device is replaced, the data structures can be transmitted, so that the history of the previous behavior of usage, based on which the simulation is made, remains the same, and the device behavior is identical with regard to user inputs. This also comprises, e.g., the presetting of operating parameters and proposals for further selection possibilities of actions in menus or certain control characteristics, i.e., response times for changes in the setpoint specifications.

1. A method for sensing and displaying the likelihood of failure of a medical-technical device,
wherein the medical-technical device comprises
at least one wear-prone component,
at least one sensor for sensing the operating parameters of the wear-prone component,
at least one memory for storing the operating parameters, the memory comprising at least one operating parameter limit,
at least one computer for calculating the likelihood of failure, and
at least one display unit,
characterized by that the computer compares the operating parameters of the medical-technical device to stored operating parameter limit data and displays the result in the form of a likelihood of failure and/or a remaining usable life and/or a number of remaining usable cycles.

2. The method of claim 1, wherein the medical-technical device is a liquid pump for laparoscopy, arthroscopy, hysteroscopy, uroscopy, or an insufflation device.

3. The method of claim 1, wherein the wear-prone component is the motor of a peristaltic pump, the gas pump of an insufflation device, or a vacuum pump of an insufflation device.

4. The method of claim 1, wherein the measured operating parameter is selected from the duration of use, the operating current, the ratio of current consumption to rotating speed, the ratio of current consumption to pressure, the ratio of rotating speed to flow rate, the maximum pressure, the maximum flow, the pressure rise per unit of time, the flow rise per unit of time, the vibration, the temperature, the noise during operation.

5. The method of claim 1, wherein measured and/or stored operating parameters are transmitted via a device interface with or without encryption to an intranet or the internet.

6. The method of claim 1, wherein the device memory contains the operating parameter limits in a latched manner, or wherein the device memory can be reloaded through a device interface.

7. The method of claim 1, wherein the calculation of the possible remaining usable life or the remaining usable cycles is performed outside the device.

8. The method of claim 1, wherein the calculation of the remaining usable life or the remaining usable cycles occurs at the beginning or the end of a medical treatment.

9. The method of claim 1, wherein the display of the remaining usable life and/or of a number of remaining usable cycles occurs on a device display, per SMS, email, and/or data transmission on a hospital information system, wherein, optionally, optical and/or acoustic alarm signals are output when the parameter limits are reached.

10. The method of claim 1, wherein, when the device is replaced, data transmission to a successor device occurs, and/or wherein, when a wear-prone component is replaced, the stored data about time or intensity of use are renewed.

11. A medical-technical device for carrying out a method of claim 1, wherein the medical-technical device comprises at least one wear-prone component,
at least one sensor for sensing the operating parameters of the wear-prone component,
at least one memory for storing the operating parameters, the memory comprising at least one operating parameter limit,
at least one computer for calculating the likelihood of failure, and
at least one display unit,
characterized by that the computer compares the operating parameters of the medical-technical device to stored operating parameter limit data and displays the result in the form of a likelihood of failure and/or a remaining usable life and/or a number of remaining usable cycles.

12. The medical-technical device of claim 11, wherein the device is a liquid pump for laparoscopy, arthroscopy, hysteroscopy, uroscopy, or an insufflation device.
